(19) United States
(12) Patent Application Publication
(10) Pub. No.: US 2026/0191447 A1
(43) Pub. Date: Jul. 9, 2026

(54) SYNCHRONIZED MULTISENSORY METHOD TO MODIFY BEHAVIORAL FUNCTIONS

(71) Applicant: Mentalness LLC, Doral, FL (US)

(72) Inventors: Tomas ORTIZ ALONSO, Madrid (ES); Julio BONIS SANZ, Madrid (ES); Alberto SOLE, Doral, FL (US)

(21) Appl. No.: 19/134,561

(22) PCT Filed: Dec. 1, 2023
(86) PCT No.: PCT/US2023/082133

§ 371 Date: May 30, 2025

Related U.S. Application Data
(60) Provisional application No. 63/429,859, filed on Dec. 2, 2022.

(51) Int. Cl. A61B 5/16; A61B 5/00

(52) U.S. Cl. CPC A61B 5/165; A61B 5/6802; A61B 5/7267

(57) ABSTRACT

A Synchronized Multisensory Method Protocol is disclosed that synchronizes using brain frequency bands virtual reality with multisensory processes to affect behavior. The Synchronized Multisensory Method Protocol uses clinical, psychological, psychosocial, functional, and sensory variables, as well as scores and scales validated in the field of health, to measure the evolution of neurocognitive learning as applied during training sessions to a user. The Synchronized Multisensory Method Protocol is a computer-based system that uses hardware, based on a set of devices available on the market to generate sensory stimuli using brain frequency bands as well as measure physiological signals that allow measuring in real time the response of the body to the stimuli programmed by the method. The system uses software linked to artificial intelligence models that measure the body's response in real time and allows customization of the protocol in terms of the various stimuli applied.

### FIELD OF THE INVENTION

The invention relates to a Synchronized Multisensory Method that synchronizes virtual reality with multisensory processes to modify behavioral functions. The Synchronized Multisensory Method uses clinical, psychological, psychosocial, functional and sensory variables, as well as scores and scales validated in the field of health, to measure the evolution of neurocognitive learning as applied during training sessions to a user. The Synchronized Multisensory Method is a computer-based system that uses hardware, based on a set of devices available on the market to generate sensory stimuli (visual, tactile, olfactory, gustatory, and auditory) as well as measure physiological signals that allow measuring in real time the response of the body to the stimuli using natural brain frequencies (delta, theta, alpha, beta and gamma) programmed by the method. The system and method use software linked to artificial intelligence models that measure the body's response in real time and allows customization of the protocol in terms of temporality, intensity, frequency, and sequence of the various stimuli applied.

### BACKGROUND OF THE INVENTION

The scientific community has conducted work in recent years on the importance of multisensory systems in neurological, psychiatric diseases and psychological and behavioral disorders, as well as in neurocognitive developmental processes. Studies of multisensory integration of auditory, visual, tactile, olfactory, or gustatory stimuli help explain how the brain functions in response to behavior. The human brain can combine what a person sees, hears, feels, smells or tastes to understand human behavior based on multisensory perception. Studies have shown that the integration of several sensory stimuli can result in richer and more intense multisensory experiences than those achieved by presenting them individually, causing a person to develop selective attentional processing that facilitates perception, learning and memory.

Multisensory integration is the process by which the nervous system synchronizes information from different sensory modalities, neurophysiological functions, and even from body prosthetics, to provide a neurofunctional response to human behavior.

Another important aspect of multisensory integration is that the availability of the same information temporally, simultaneously, and synchronously through two or more sensory systems is a common natural process. This ability of the brain to integrate several “redundant” sensory systems over time is conducive to an increase in attention span and facilitation of perception, learning and memory.

One way of integrating multisensory systems is through virtual, augmented, and extended reality (VR, AR, XR) which is a tool capable of synchronizing sensory, physiological, cognitive and emotional processes for human behavior modification. VR, AR and XR makes it easy for a person to follow and adhere to the stimuli in a pleasant and highly motivated way. VR, AR and XR is understood as a real experience through a fictitious world generated by a computer that allows the person to immerse in a programmed environment and interact through perception, imagination, and action, giving the sensation of being in a totally real environment.

VR/AR/XR allows the creation of an illusory mental state simultaneous to a normal state of consciousness and wakefulness. It provides multisensory, proprioceptive, and motor information within a virtual reality scenario that, in a synchronized manner, gives the user the sensation of being immersed. VR/AR/XR generates a subjective experience through two processes, one of immersion, which is a psychological state in which one perceives oneself in an active sense; and passive, in an environment characterized by the continuity of stimuli and events (what, how much and how is perceived) and the other, through interaction, which is a psychological state in which attention is focused on a set of stimuli, activities and events (what one does, how one does it and how much one does it).

Through VR/AR/XR and multisensory synchronization, the human brain can be trained to develop new behaviors that diminish the effects of psychiatric disorders such as anxiety, phobias, obesity, post-traumatic stress and others, as well as to improve certain cognitive functions that have deteriorated over time, such as attention, perception, memory, learning; or motor functions, such as precision, adaptation or execution of movements.

The combination of virtual and multisensory reality can enhance treatment in patients who do not respond to standardized treatments and memory recovery. VR/AR/XR makes it possible to determine relevant multisensory stimuli capable of increasing emotional sensitivity in patients with post-traumatic stress, enabling better and faster recovery. Multisensory stimuli, such as taste, smell, sight, touch, and hearing, are directly involved in food perception and intake, are important factors inducing overweight or obesity.

In relation to cognitive processes, over time, deteriorations are observed in mental functions, such as selective attention, information processing speed, working memory, response inhibition, prospective memory, and episodic memory. These losses have been associated with alterations in the multisensory processes of information input to the brain such as sight, hearing, touch, taste, smell or simple processes of motor organization, proprioception or biological balance in ingestion and hydration. Rejuvenation in old age takes multisensory factors into account. The use of VR/AR/XR and interaction with multisensory stimuli has been noted in the improvement of sports motor response and in physical processes such as gait and balance in older people, in people with post-traumatic stress disorder and in patients with anxiety disorders.

### BRIEF SUMMARY OF THE INVENTION

The Synchronized Multisensory Method Protocol (SMM Protocol) according to the invention comprises a brain learning protocol of modifying human functional behavior by synchronizing VR/AR/XR and multisensory stimuli using traditional frequency bands with neurophysiological, cognitive, and emotional processes.

In one embodiment the SMM Protocol is conducted via a learning protocol through training in which visual, auditory, tactile, olfactory, and gustatory stimuli are synchronized through VR/AR/XR with neurophysiological, cognitive, and emotional processes typical of human behavior. The different elements or events used by the SMM Protocol, both at the level of virtual, augmented, or extended reality and simultaneous stimuli of touch, smell, taste, and/or physical exercises, can be adapted by means of artificial intelligence models that compensate for and improve the learning method applied in a personalized way. The SMM Protocol can be executed through daily sessions to modify or correct behavioral functions.

In one embodiment, to carry out the SMM Protocol, wearable devices are used by a user in each training session to synchronize visual, tactile, olfactory, and gustatory stimuli, associated with VR/AR/XR using brain frequency bands with the goal of influencing the behavioral functions of people.

In one embodiment, a physiological and physical signal capture wearable device is worn by the user during each training session that measures the user's real-time response to various phases of multisensory stimuli. Using an Optimization Artificial Intelligence Model based on the use of artificial intelligence (AI), those stimuli's interactions with the user can be modified for future training sessions in terms of frequency, temporality, and intensity, as well as synchronization between the different stimuli.

The SMM Protocol allows the measured learning progress of the user to analyze the evolution and modification of one or more behavioral functions. The SMM Protocol is believed to promote the reorganization of new neural circuits because of regular training over time, which will allow the user to modify not only their altered behaviors, cognitive, social, or emotional processes, but also to improve their neuropsychological functions.

### BRIEF DESCRIPTION OF THE DRAWINGS

The various features of the invention and the manner of attaining them will be described in greater detail with reference to the following description, claims, drawings, wherein like designations denote like elements.

FIG. 1 depicts an execution flowchart of clinical, psychological, psychosocial, functional, and sensory evaluation of the user in Category 1 of the SMM Protocol according to one embodiment of the invention.

FIG. 2 depicts a schematic of a Catalog of Methods and Contents of Multisensory Stimulation and use of the catalog in a Multisensory Stimulus Synchronization Process used by software to run multisensory stimulus elements during execution of the SMM Protocol.

FIG. 3 is a schematic and flow scheme of the process of the Adaptive Stimulation Program Orchestration System for neuro stimuli based on control by the Optimization Artificial Intelligence Model.

### DETAILED DESCRIPTION OF THE INVENTION

According to one embodiment of the invention, the SMM Protocol comprises a brain learning protocol of modifying human behavioral functions by synchronizing VR/AR/XR and multisensory stimuli using brain frequency bands (delta, theta, alpha, beta and gamma) with neurophysiological, cognitive, and emotional processes conducted via a learning protocol through training in which visual, auditory, tactile, olfactory, and gustatory stimuli are synchronized through VR/AR/XR with neurophysiological, cognitive, and emotional processes typical of human behavior. The SMM Protocol comprises two parts:
The first part of the SMM Protocol comprises a technological platform that includes methods and contents of multisensory stimulation, input, and output wearable devices to measure a user's body to stimuli, software for synchronization of a variety of multisensory stimuli and an AI model that is programmed to provide modification of the frequency, intensity and synchronization between different multisensory stimuli based on physiological response signals of the user to stimuli.
The second part of the SMM Protocol uses multisensory stimulation synchronized with virtual reality carried out through regular sessions, each one of them with the purpose of modifying behavioral functions comprising general behavior and/or specific cognitive or emotional abnormal behaviors.

The SMM Protocol, both at the level of virtual, augmented, or extended reality and simultaneous stimuli of touch, smell, taste, and/or physical exercises, can be adapted by the Optimization Artificial Intelligence Model to improve and/or enhance learning applied during training sessions in a personalized way.

In one embodiment, to carry out the SMM Protocol, wearable devices are worn by a user in each training session to synchronize visual, tactile, olfactory, and gustatory stimuli, associated with VR/AR/XR with the goal of influencing the behavioral modification of people.

In one embodiment, a physiological and physical signal capture wearable device is worn by the user during each training session that measures the user's real-time response to various phases of multisensory stimuli. User responses to various stimuli can be recorded and analyzed according to the Optimization Artificial Intelligence Model, wherein the SMM Protocol can be modified according to the Optimization Artificial Intelligence Model for future training sessions in terms of frequency, temporality, and intensity, as well as synchronization between the different stimuli. In one embodiment, the SMM Protocol can be modified according to the Optimization Artificial Intelligence Model in real-time during a training session.

The SMM Protocol allows the measured learning progress of the user to analyze the evolution of one or more behaviors. The SMM Protocol is believed to promote the reorganization of new neural circuits because of daily training over time, which allow the user to modify not only their behavior functions, cognitive, social, or emotional processes, but also to improve their neuropsychological functions.

One or more physiological parameters of a user comprising pulse, heart rate, respiratory rate, SpO₂, ECG and combinations thereof are measured during each training session of the SMM protocol, which physiological parameters are gauged at the end of each training session to construct a scale (the “SMM Scale) to compare the physiological parameters of the user over time with previous and initial values of those physiological parameters.

In one embodiment, the SMM Protocol is conducted with a user in daily training sessions. In one embodiment, the SMM Protocol is conducted with a user in training sessions in intervals other than daily sessions. In one embodiment, the SMM Protocol is conducted with a user in training sessions held over a three (3) month period. In one embodiment, the SMM Protocol is conducted with a user in training sessions held for less than three (3) months. In one embodiment, the SMM Protocol is conducted with a user in training sessions held for more than three (3) months.

Five categories are used in the two parts of the SMM Protocol noted above.

### Category 1: Synchronized Multisensory Method Protocol.

The SMM Protocol measures medical, psychological, psychosocial, and functional responses of a user in validating an AI learning progress for use in modification of the user's behavioral functions over time using control variables established in an initial evaluation phase. For this purpose, at the end of the training period of the SMM Protocol, values of various control variables are taken and compared with the initial and prior training sessions, in order to assess the difference and evolution of the control variables throughout the training sessions of the SMM Protocol.

In Category 1, the SMM Protocol begins with a medical, psychological, psychosocial, functional, and sensory evaluation of a user as control variables to define the user's initial status and to determine the behavioral function desired to be controlled and/or modified. The values of these control variables are used to construct an SMM Scale so that measurements of the same control variables can be compared after training sessions, allowing for evaluation of the behavioral function sought to be controlled and/or modified, as well as physiological and psychological processes.

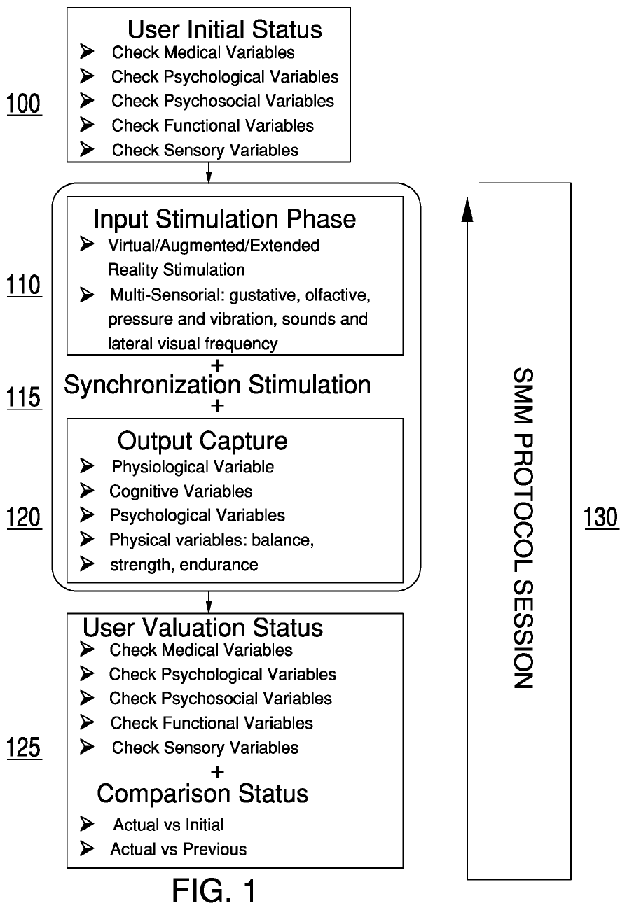

An execution flowchart of one embodiment of the medical, psychological, psychosocial, functional, and sensory evaluation of control variables of the user in Category 1 of the SMM Protocol is depicted in FIG. 1.

At 100, a variety of measurements of control variables comprising medical control variables, psychological control variables, psychosocial control variables, functional variables, sensory variables, or combinations thereof are made on the user prior to conducting a first training session of the SMM Protocol to provide an initial status. The control variables to be measured may include other variables not mentioned or may not include only some of these variables as desired by the operator of the SMM Protocol which may be determined according to the desired behavioral function to be controlled and/or modified.

The medical control variables may include but are not limited to the following:
Physical examination (mobility, speech disturbances, gait, hydration, skin color).
Vital signs (body temperature, pulse, respiratory rate, blood pressure).
Personal history (family history, previous pathologies, possible surgical interventions, type of previous medication).
Anthropometric variables: Height, Weight, Body Mass Index, folds, fat percentage, lean and fat reserve, bone mass and residual mass, weight index and waist/hip ratio.
Nutritional variables: biochemical parameters (glucose, cholesterol,) and dietary indicators, such as knowledge of food consumption, food habits, frequency, and preferences.
Endocrinological variables: ghrelin, leptin, insulin, sex hormones, cortisol, catecholamines, prolactin, growth hormone (GH), thyroxine.
Physical activity (very active, active, sedentary).
Pharmacology and supplements (type and quantification of medication and/or supplements taken by the user).

The psychological control variables may include but are not limited to the following:
Mental State
Attention
Memory
Depressive State
Anxiety Levels
Stress
Frustration
Self-esteem
Personality (personality traits)

The psychosocial control variables may include but are not limited to the following:
Educational level
hobbies
physical activity
Type of lifestyle (active to sedentary)
Environmental variables: work, cultural level, lifestyle, hobbies, physical activity
Sleep/wake cycle patterns

The functional control variables may include but are not limited to the following:
Gait
Balance
Gait Speed
Muscle Strength and tension
Grip strength

The sensory control variables may include but are not limited to the following:
Visual
Auditory
Tactile
Olfactory
Gustatory

At 110, a first training session is conducted with the user. Visual, auditory, tactile, taste, and olfactory input elements (sensory stimuli) that may be used and synchronized using brain frequency bands during training sessions of the SMM Protocol may comprise the following:
Visual: ocular motility or photic stimulation at different frequencies band.
Auditory: stimulation of tones, sounds at different frequencies band.
Touch: vibrational or pressure frequency at different frequencies band
Taste: different flavors in relation to visual and video stimuli band.
Smell: different smells in relation to visual and video stimuli band
Positive images of oneself young and with normal weight band.
Multisensory synchronization to certain stimuli of the Virtual, Augmented or Extended Reality video

At 115, the visual, auditory, tactile, taste, and olfactory input elements are synchronized at brain frequency bands (delta, theta, alpha, beta and gamma) with VR/AR/XR images or scenes associated with the behavioral function sought to be modified and/or controlled. Synchronization of sensory stimuli takes place at the beginning of an image or scene presented via VR, AR, or XR. In one embodiment, the duration of the synchronization of the sensory stimuli will be approximately the same as the image or scene presented via VR, AR, or XR associated with the behavioral function sought to be modified or controlled.

In one embodiment, each training session will comprise five VR, AR, or XR videos. The first VR, AR, or XR video is an introduction to the SMM Protocol, which will inform the user about the purpose of the training session, directed to generating strength or motivation in the user. The second VR, AR, or XR video will be devoted to a relaxation process using images and scenes associated with multisensory stimulation. The third VR, AR, or XR video will comprise a series of physical exercises associated with breathing and multisensory stimulation to fine-tune the body of the user. The fourth VR, AR, or XR video will be dedicated to the presentation of images linked to the behavioral function that is sought to be corrected or modified and will be synchronized with sensory stimulation. The fifth VR, AR, or XR video will focus on the context and exposure of scenes related to the behavioral function sought to be corrected and/or modified synchronized with multisensory stimuli. Finally, the training session will end with the re-presentation of the second VR, AR, or XR video to keep the user in a state of relaxation at the end of the training session. In other embodiments, the number of VR, AR, or XR videos; the sequence of the VR, AR, or XR videos; and the subject of the VR, AR, or XR videos may be selected as desired by the operator of the SMM protocol.

At 120, measurements of functional, cognitive, psychological, and physiological response signals of the user to sensory input elements including sensory stimuli and VR, AR, or XR videos are recorded during each training session to obtain the response of the body of the user to the various multisensorial inputs. The responses of the user's body output signals are analyzed by the Optimization Artificial Intelligence Model to allow for customization of the SMM Protocol at the level of sequence, frequency, temporality, and intensity of the multisensory stimuli. The Optimization Artificial Intelligence Model may analyze the response values in real-time or after each training session.

Examples of different variables and parameters used in the body response control process include but are not limited to the following:
Physiological parameters: pulse, heart rate, breathing rate, SpO₂, and ECG.
Cognitive parameters: memory of synchronized images with multisensory stimuli; and sensory perceptual discrimination, selective attention to certain stimuli of the protocol. These can be executed during the multisensory stimulation synchronization protocol or at the end of it, depending on the result of the different sessions performed.
Psychological parameters: emotional state, degree of satisfaction. These can be executed during the multisensory stimulation synchronization protocol or at the end of it, depending on the result of the different sessions performed.

At 125, after the end of each training session, the user is evaluated for the same control variables as initially done and an SMM Scale can be constructed comparing the values of the control variables over time as more training sessions are conducted.

Variables that can be measured to ascertain the effectiveness of a training session may comprise the following:
Anxiety
Self-esteem.
Attention and Memory
Cortisol (stress)·
Body Mass Index
Improvement of eating habits
Balance
Gait Speed
Sleep/wake cycle patterns
Physical activity
Type of lifestyle (sedentary)

In one embodiment, each training session lasts about 15 minutes, and physiological variables (pulse, heart rate, respiratory rate, SpO₂ and ECG) are monitored throughout.

At 130, following analysis by the Optimization Artificial Intelligence Model to see the effectiveness of behavioral learning and to modify at the individual level the frequency, changes to the multisensory inputs may be made for subsequent training sessions at 110.

In one embodiment, the user may be asked to take a satisfaction survey.

### Category 2: Catalog of Methods and Contents of Multisensory Stimulation (MCMS).

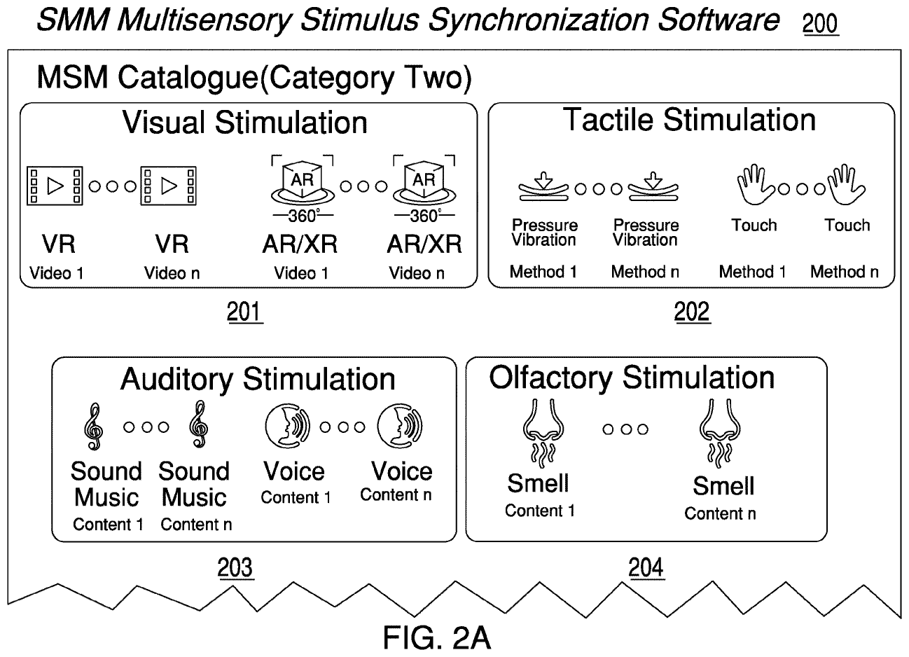

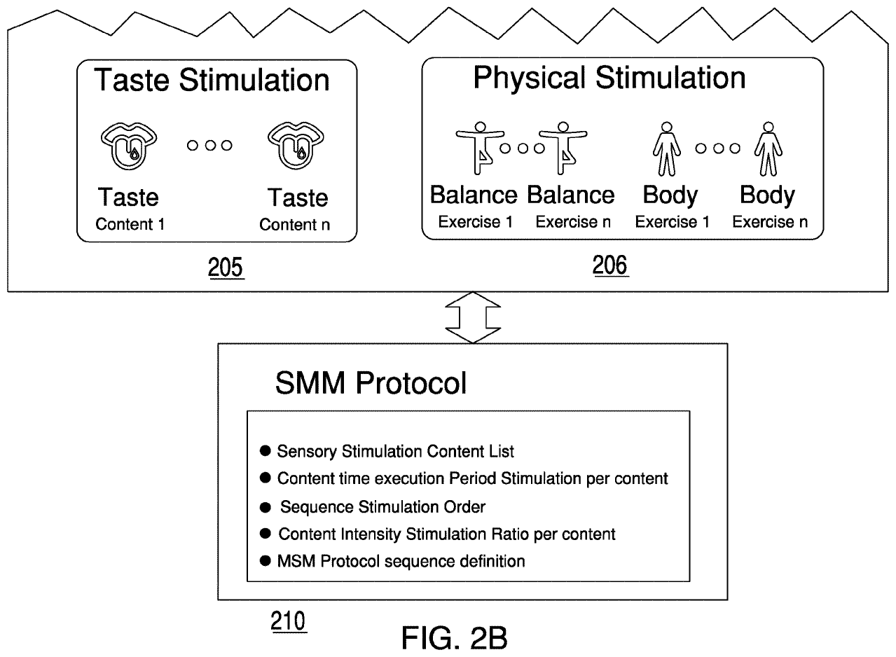

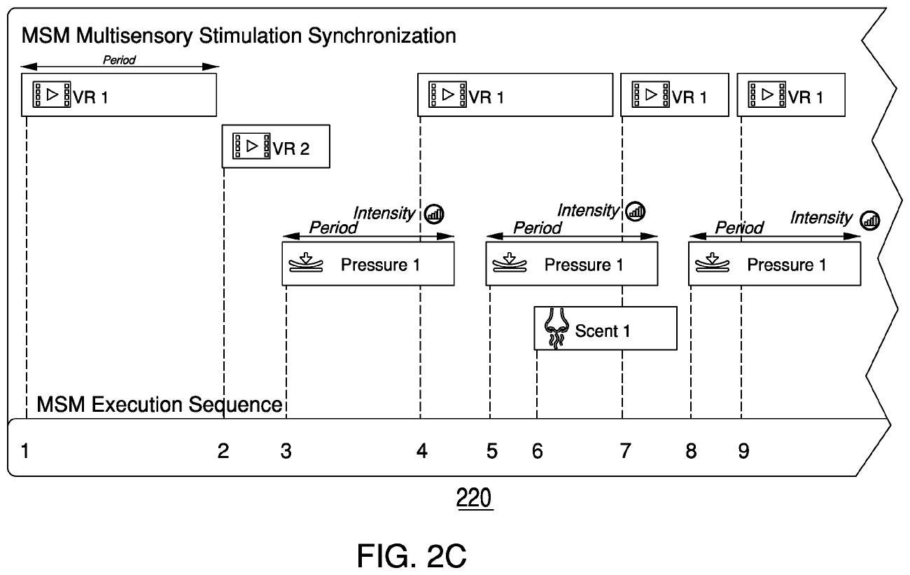

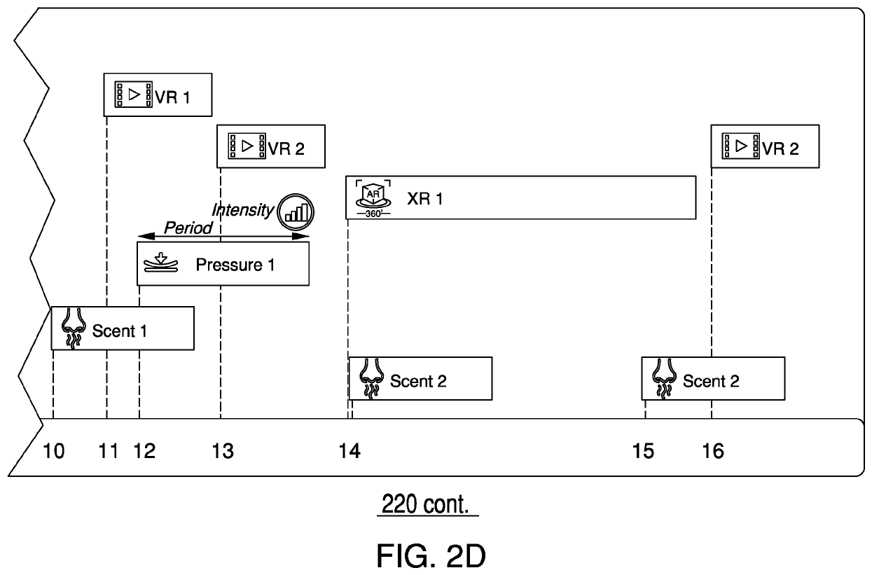

In Category 2, a Methods and Contents of Multisensory Stimulation (MCMS) catalog is created as seen in FIG. 2 at 200 for storing VR, AR, or XR videos at 201 along with files relating to other sensory stimuli including but not limited to tactile stimulation such as pressure vibration and touch at 202; auditory stimulation such as sound, music and voice at 203; olfactory stimulation at 204; taste stimulation at 205; and physical stimulation at 206. This collection of sensory stimulus elements is stored in a database, where synchronization software between VR/AR/XR files and multisensory stimulation files allows files to be extracted and executed during each training session. The methods and contents of multisensory stimulation files are classified and labeled with families of objects to be used during a training session according to certain criteria and properties, including for example the following:
Families associated with methods and contents:
   Visual Sense: Virtual Reality, Augmented or Extended Reality, lateral visual stimulation.
   Olfactory sense: olfactory stimulus
   Gustatory Sense: gustatory stimulus
   Sense of Touch: localized or generalized pressure and touch stimuli
   Auditory Sense (sounds, songs, voice commands).
Properties applied to methods and contents:
   Visual Sense: temporality of the visual stimulus that appears in the scene of Virtual Augmented or Extended Reality, intensity that is associated with the level of image (pleasant or unpleasant) as well as frequency used for lateral stimuli.
   Olfactory sense: temporality of the olfactory stimulus associated with pleasant or unpleasant odors, intensity of the odor.
   Gustatory Sense: temporality of the gustatory stimulus associated with pleasant or unpleasant flavors, intensity of the flavor.
   Sense of Touch: temporality of the tactile stimulus or pressure exerted on certain body parts, intensity of touch in friction or temperature as well as localized pressure, and frequency of tactile or pressure stimuli to intensify the stimulus.
   Auditory Sense: temporality of the auditory stimulus associated with pleasant or unpleasant sounds, songs, or voice commands, sound intensity using a specific sound frequency range to obtain emotional states in the individual such as relaxation, tension, overwhelm, euphoria, joy, peace.

### Category 3: Multisensory Stimulus Synchronization Software.

In Category 3, Multisensory Stimulus Synchronization Software (MSSS) is developed based on guidelines established by the SMM Protocol. The order of execution and temporality of multisensory stimuli stored in the MCMS catalog established in Category 2 at 200 are specified, as are the intensity derived from the intrinsic properties of each sensory input (touch, auditory, visual, olfactory, and gustatory senses), and the frequency of use of various multisensory stimuli during execution of the SMM Protocol at 210 in FIG. 2. Multisensory stimulus elements used in the training sessions are synchronized by the MSSS allowing execution of the SMM Protocol in a programmed way that will allow for automatic learning.

As seen at 220 in FIG. 2, MSSS extracts from the MCMS elements in image, video, sound, or object format of stimulus to be carried out during a training session. These elements are carried out by various wearable technology devices, which enable the emission of sensory stimulus signals from the user who is wearing the wearable technology devices via connection interfaces with the wearable technology devices or by audio and video hardware in the case of VR/AR/XR files. For each training session, the various wearable technology devices and hardware are fixed and adapted to execute desired elements from the MCMS (such as but not limited to hearing aids, glasses, taste and smell equipment, and tactile stimulation equipment).

MSSS allows synchronization using brain frequency bands of a wide variety of multisensory stimuli for execution in training sessions. MSSS further allows the capture and recording of various synchronizations of multisensory stimuli elements during training sessions to be stored in a database. MSSS processes the various multisensory stimuli elements' inputs as well as the real-time reading of physical, psychic, and functional responses of the user to these various multisensory stimuli elements. This enables the Optimization Artificial Intelligence Model to access the various synchronizations of multisensory stimuli elements and construct new execution sequences that MSSS will carry out during training sessions. Accordingly, MSSS is the orchestrator that allows the SMM Protocol to be executed during a training session in an efficient and controlled way of all the stimuli that are utilized during the training sessions, as well as the execution of the synchronized reading in real time of the physiological constants and the functional, cognitive, and psychological parameters of the user's response to these stimuli. FIG. 2 at 230 summarizes the synchronization process of various stimulus elements by MSSS software to run the SMM protocol.

### Category 4: Optimization Artificial Intelligence Model Based on an Adaptive Stimulation Program Orchestration System.

An Adaptive Stimulation Program Orchestration System comprises a set of software modules capable of generating random stimulation programs using the Optimization Artificial Intelligence Model that, when applied to groups of people, allow the identification of combinations suitable for each user of the SMM Protocol based on stimulation program (SP) sessions that are carried out by the Optimization Artificial Intelligence Model.

### Components of an SP.

An SP is made up of a series of “activities,” A (A1, A2, . . . . An), that are executed in a specific order and timed by a programmable computer. Each activity is composed of a combination of “stimuli” S {S₁, S₂, . . . , Sₙ} configured to have a duration taken from a finite set of “durations” D {D₁, D₂, . . . . Dₙ} measured in milliseconds and an intensity taken from a finite set of “intensities” I {I₁, I₂, . . . . Iₙ}. A fundamental attribute of the Adaptive Stimulation Program Orchestration System is that all activity is composed of multimodal “stimuli” being the modalities: auditory, visual, tactile, and olfactory. Different hardware devices can be connected to the Adaptive Stimulation Program Orchestration System to generate stimuli that make up a given activity such as virtual reality glasses, headphones, smart clothes, and others. There is a set of “results” O {O₁, O₂, . . . . Oₙ} that can be measured with a configurable frequency F by means of different hardware devices and sensors.

The results or outcomes O can be defined as surrogate outcomes O^{s} or target outcomes Ot. A surrogate outcome is a measurement, typically physiological (as heart rate), that reflects the immediate effect of a stimulus on the organism and can be used as short-term evidence of the effectiveness of the activity. A target outcome defines the real long-term objective for a SP, as for example the weight of an individual or the average duration of sleep.

### Adaptive Stimulation Program Orchestration System.

The Adaptative Stimulation Program Orchestration System supports two options to generate a SP.

The first option is based on the manual definition of an SP by human domain experts through the definition of a specific set of activities A, whose ordered sequence and time schedule is manually defined by the human operator, as well as a surrogate outcome.

During the execution of an SP defined by a human operator in a particular user a module titled “AI Customizer” is programmed to adjust the duration D and intensity I of each stimuli S so the physiologic measure O^{s} in the user reaches a desired value. For example if one of the stimuli of the manually defined SP is an acute noise and during the programming of the SP it was established as a physiological outcome a heartbeat frequency over 100 beats per minute, the AI Customizer is programmed to adjust iteratively the intensity I and duration D of the noise stimulus to each particular user so that a heartbeat of at least 100 beats per minute is achieved.

The second option for the generation of a SP is based on a Neurostimulation Program Generator (NSP Generator) which comprises programmed software that runs on a general purpose computer. The NSP Generator is a module comprises a sequence (randomly generated) of activities (with random durations and intensities) accompanied by a fixed target outcome O^{t} measurement plan. An activity could be adversive or reenforcing, as for example: “visualization of junk food combined with tactile and auditory discomfort stimuli” or “visualization of eating healthy food combined with pleasant tactile stimuli or other visually positive stimuli. The NSP Generator randomly generates a group of N different SPs from an initial configuration file defined by human operators, wherein the initial configuration file comprises a list of available activities A, durations D, intensities I and outcomes) with randomly selected variations of A-D-I sequences and a fixed O measurement plan, and passes them to a A/B Test Allocator module. The A/B Test Allocator module is programmed to run on a general purpose computer. The A/B Test Allocator module assigns a different sequence of Activities A, B, C etc. having different types, intensities, and durations of aversive and reinforcement stimuli based on demographics and other user profile variables according to predetermined conditions and parameters.

The fine tuning of the NSP Generator begins initially with the random generation of activity-duration-intensity sequences that in a first phase are randomly assigned to users of the SMM Protocol. At this point there is no knowledge about which activity-duration-intensity sequences are associated with better or worse outcomes (each SP has a different outcome metric defined). At the beginning of the fine-tuning process each activity-duration-intensity sequence randomly generated is assigned with a numerical value (weight) as determined by the operator according at least in part with regard to a behavioral function to be modified by the SMM Protocol. During the fine tuning (FIRST PHASE) the activity-duration-intensity sequences that are followed by better outcomes for effectiveness on an activity are assigned with higher weights by the operator and thus are assigned to the users with a higher probability of success of effecting that activity. An Adaptative Recommendation Model that is programmed to run on a general-purpose computer adjusts the weights (previously adjusted during the FIRST PHASE) again during a SECOND PHASE of the NSP Generator fine-tuning process in order to improve the desired outcome.

The A/B Test Allocator module runs an “Experiment” assigning each user of the SMM Protocol a specific variation of SP activities generated by the NSP Generator Module over time based on the demographic characteristics of a group of users (age, gender, location, medical conditions, previous values for measured results). The A/B Test Allocator module determines when to end each SP run and when to assign new SP activity variations to users according to predetermined criteria. The Experiment can be defined in a configuration file that includes inclusion criteria (what characteristics the user must have to be included in the experiment), exclusion criteria (which features will prevent a user from being included in the experiment), censorship criteria (what characteristics will cause a user to be excluded from the experiment once it has been included), the results measurement plan and branch assignment criteria (how to assign users to different SP variants, if completely random, using a random stratified process, or how to switch users between branches).

An Adaptive Recommendation Module programmed to run on a general purpose computer analyzes the results generated by different Experiments, identifying which variations of SP activities have the greatest impact on different results in different subpopulations of users and thus predicting the expected impact on the result variables based on the demographic characteristics of a specific SMM Protocol user including the previous history of variants executed on that user and the impact of the result obtained. Based on that prediction, the Adaptive Recommendation Module provides recommendations on a variant of SP activities to run during the SMM Protocol to get a desired result.

In a SECOND PHASE, once an initial weight adjustment of activity-duration-intensity sequences has been obtained according to the desired result of the SMM Protocol for a specific user, the Adaptive Recommendation Model adjusts the weights of the activity-duration-intensity sequences to improve the desired result (the target function) for each user and thereafter recommends a revised sequence of activities, types, intensities, and durations for SPs.

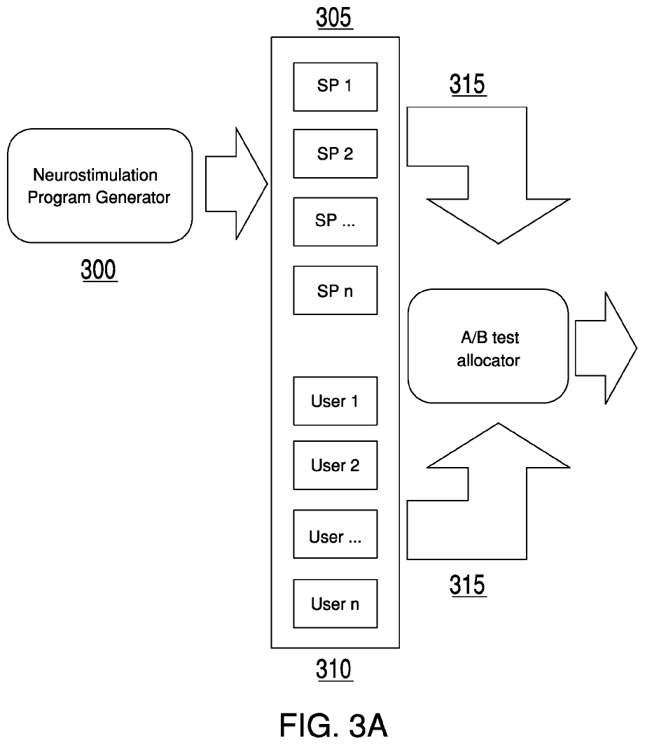

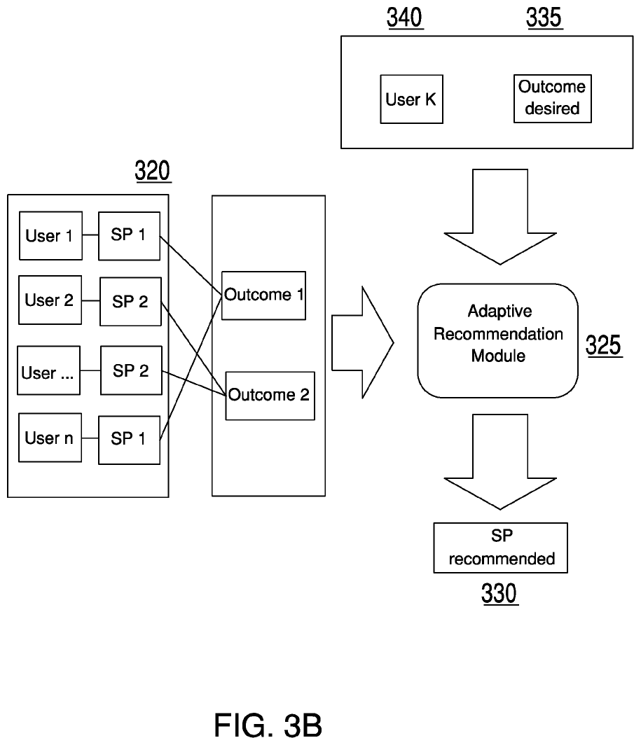

FIG. 3 depicts the process carried out by the Adaptive Stimulation Program Orchestration System. At 300, the NSP Generator has been previously composed of a sequence (randomly generated) of activities (with random durations and intensities) that could be adversive or reenforcing. The NSP Generator randomly generates at 305 a group of N different neurostimulation programs (SP 1, SP 2, SP . . . , SP n) from an initial configuration file, wherein the initial configuration file comprises a list of available activities A, durations D, intensities I and outcomes O with randomly selected variations of A-D-I sequences and a fixed O measurement plan. The NSP Generator further comprises a file 310 of user profiles User1, User2, User . . . , Userₙ. At 315, the NSP Generator passes the generated SPs to an A/B Test Allocator module which assigns a different sequence of Activities A, B, C etc. having different types, intensities, and durations of aversive and reinforcement stimuli based on demographics and other user profile variables according to predetermined conditions and parameters. At 320, the A/B Test Allocator runs a plurality of Experiments assigning each Userₙ a specific variation of an SP activity SP n generated by the NSP Generator Module [over time based on the demographic characteristics of a group of users (age, gender, location, medical conditions, previous values for measured results). At 325, the Adaptive Recommendation Module analyzes the results generated by different Experiments, identifying which variations of SP activities have the greatest impact on different results in different subpopulations of users and predicting the expected impact on the result variables based on the demographic characteristics of a specific SMM Protocol user including the previous history of variants executed on that user and the impact of the result obtained. Based on that prediction, at 330 the Adaptive Recommendation Module provides recommendations on the best variant of SP activities to run during the SMM Protocol to get a desired result 335 for a specific user Userₖ at 340.

The Optimization Artificial Intelligence Model allows for adjustment of the properties of sequence, frequency, and intensity of various SPs to optimize the SMM Protocol.

### Category Five: The Measurement of Results.

The measurement of results of the SMM Protocol obtained at the end of each training session is based on the SMM Scale that integrates a list of psychological processes, motor functions, anthropometric assessments, and nutritional profiles. The SMM Scale combines scales and methods that have been established and scientifically validated in the market, and it employs a mathematical method that uses the weight of each of the elements listed below to define an integral model that allows measurement of the sensory multi-stimulation response.

### Psychological Processes:

Trait Anxiety: detect people who have a predisposition, both genetic and environmental, to suffer from an anxiety disorder.
State Anxiety: analyze the immediate emotional state, transitory and modifiable over time.
Self-esteem: analyze the positive or negative evaluation that the user makes of himself/herself.
Depression: assess the state in which the user is on the depression scale.
Body image assessment: analyze the concept that each person has of his or her own physical appearance.
Attention: analyze the ability to generate, direct and maintain a short active functional state during information processing.
Memory: analyze the functional capacity that enables a user to record, preserve and recall experiences.
Personality traits: analyze the set of characteristics, emotions, ways of thinking and behavioral aspects of an individual that serve to define and value him/her as stable traits over time.

### Physiological Processes:

Cortisol: measure cortisol which is a hormone usually associated with stress, states of anxiety, distress, or emotional tension.
Catecholamines: measure the release of catecholamines, which are neurotransmitters associated with states of stress and anxiety.
Prolactin: measures the release of prolactin which is a hormone associated with emotional states of sadness.
Ghrelin: measure ghrelin levels, which is a gastric hormone that regulates appetite and nutritional homeostasis and modulates physiological processes such as insulin secretion or memory.
Leptin: measure the levels of leptin which is a hormone that promotes the reduction of energy intake by signaling satiety in the brain.
Insulin: measure the release of insulin which is the hormone that controls the amount of sugar in the blood by storing it in the cells.
Growth hormone: measure the release of growth hormone which stimulates protein synthesis in muscle and the secretion of fatty acids from adipose tissue (anabolic effects).
Thyroxine: measure thyroxine which is the hormone whose function is to increase the rate of chemical reactions in cells and helps control growth and development

### Functional Processes:

Gait and Gait Speed: measure gait speed since it is associated with the fragility of the physiological state closely associated with stress factors.
Balance: measure the ability to maintain the orientation of the body and its parts in relation to outer space through balance.
Mobility: measure the range of motion of a joint, which will depend on both the state of the joint and the surrounding musculature.
Endurance: the ability of a muscle or group of muscles to perform repeated contractions against resistance for an extended period.
Flexibility: measure the ability of the muscles to adapt to varying degrees of joint movement.
Reaction time (response to external stimuli): measure the amount of time that elapses from the time we perceive something until we respond accordingly.
Agility: measure physical ability that allows individuals to decelerate quickly and efficiently, change direction and accelerate rapidly, to react appropriately to activity-relevant cues.
Coordination is the ability to perform synchronized movements efficiently, accurately, quickly, and orderly.
Muscle Strength and Tension relates to the ability to move and lift objects. intimately linked to muscle mass growth

### Anthropometric Assessments:

Body Mass: calculate the Body Mass Index (BMI) is a number that is calculated based on a person's weight and height.
Body Fat: measure the amount of fat in a person's body.
Physical Performance: analyze the improvement in physical performance.
Waist/hip ratio: measure the ratio of waist circumference to hip circumference. The ratio of fat stored in the abdominal area to that stored in the hip region is an important indicator of risk for several conditions.
Waist circumference. measure waist circumference
Waist/height ratio: calculate your waist/height ratio to detect cardiovascular and metabolic risk.
Bone mass: measure bone density to assess osteopenia and osteoporosis.
Residual mass: measure the residual weight of body components, excluding fat, muscle, and bone.
Weight Index: measure the ideal weight of everyone.

### Nutritional Variables:

Total energy profile: total energy content of foods consumed, as provided by the major sources of dietary energy:
Number of meals and their caloric distribution: how to divide meals and calories throughout the day.
Macronutrients: Macronutrients are the nutritive components of food that the body needs for energy and to maintain the body's structure and systems. There are three types of macronutrients: carbohydrates, proteins, and fats.
Micronutrients: a chemical element or substance required in trace amounts for the normal growth and development of living organisms.
Nutrient density: amount of selected nutrients per reference amount of food.
Food variety: different types of foods from the entire range of food groups like vegetables, fruit, cereals, meat, fish, and dairy products
Fluid intake: amount of water consumed from foods, plain drinking water, and other beverages.
Diet adherence: degree to which an individual ‘sticks’ to a diet.
Biochemical nutritional status: checking levels of nutrients in a person's blood, urine, or stools.
Food consumption habits: conscious, collective, and repetitive behaviors, which lead people to select, consume, and use certain foods or diets, in response to social and cultural influences.
Food consumption preferences: evaluative attitudes that people express toward foods.

### Psychosocial Variables:

Hobbies: define activities done purely for pleasure during free time.
Daily physical activity: define the physical activities done during the day.
Type of sedentary lifestyle: time spent in a sedentary lifestyle or doing little or no exercise.
Sleep/wake cycle patterns: quality of sleep which involves both a subjective assessment, as well as quantitative aspects such as sleep duration, sleep latency or the number of nighttime awakenings and purely subjective qualitative aspects such as the depth of sleep or the ability to repair it.

List of Behavior States Variables functions with their Weight assigned for SMM Scale formulation and the percentage of improvement obtained by SMM Protocol execution:
Anxiety State (Table A)
Post-traumatic stress disorder (Table B)
Cognitive rejuvenation (Table C)
Obesity, overweight (Table D)

TABLE A
ANXIETY STATES

| | | MSM βᵢ | % |
|---|---|---|---|
| Function | Test, Method, Score or Scale | Weight | Improvement |
| Psychological | Trait Anxiety | 5 | Up to 80% |
| variables | State Anxiety | 5 | Up to 80% |
| | Self-esteem | 4 | Up to 70% |
| | Depression | 4 | Up to 70% |
| | Body image assessment | 4 | Up to 70% |
| | Attention | 4 | Up to 70% |
| | Memory | 4 | Up to 70% |
| | Personality traits | 4 | Up to 70% |
| Physiological | Cortisol | 5 | Up to 80% |
| variables | Catecholamines | 4.5 | Up to 75% |
| | Prolactin | 4.5 | Up to 75% |
| | Ghrelin | 4 | Up to 70% |
| | Leptin | 4 | Up to 70% |
| | Insulin | 4 | Up to 70% |
| | Sex hormones | 4 | Up to 70% |
| | Growth hormone (GH | 3.5 | Up to 65% |
| | Thyroxine | 3.5 | Up to 65% |
| Functional | Gait and Gait Speed | 3 | Up to 60% |
| variables | Balance | 4 | Up to 70% |
| | Mobility | 2.5 | Up to 50% |
| | Endurance | 3.5 | Up to 65% |
| | Flexibility | 2.5 | Up to 50% |
| | Reaction time (response | 4.5 | Up to 75% |
| | to external stimuli) | | |
| | Agility | 3 | Up to 55% |
| | Coordination | 4 | Up to 70% |
| Anthropometric | Body Mass | 2 | Up to 40% |
| assessments | Body Fat | 2 | Up to 40% |
| | Muscle Mass | 2 | Up to 40% |
| | Physical Performance | 2 | Up to 40% |
| | Waist/hip ratio | 2 | Up to 40% |
| | Waist circumference | 2 | Up to 40% |
| | Waist/height ratio | 2 | Up to 40% |
| | Bone mass | 2 | Up to 40% |
| | Residual mass | 2 | Up to 40% |
| | Weight Index | 3 | Up to 55% |
| Nutritional | Total energy profile | 2 | Up to 40% |
| variables | Number of meals and their | 2 | Up to 40% |
| | caloric distribution | | |
| | Macronutrients | 2 | Up to 40% |
| | Micronutrients | 2 | Up to 40% |
| | Nutrient density | 2 | Up to 40% |
| | Food variety | 2 | Up to 40% |
| | Fluid intake | 2 | Up to 40% |
| | Diet adherence | 2 | Up to 40% |
| | Biochemical nutritional status | 2 | Up to 40% |
| | Food consumption habits | 2 | Up to 40% |
| | Food consumption preferences | 3 | Up to 55% |
| Psychosocial | Hobbies | 2 | Up to 40% |
| variables | Daily physical activity | 4 | Up to 70% |
| | Type of lifestyle | 5 | Up to 80% |
| | (active to sedentary) | | |
| | Sleep/wake cycle patterns | 5 | Up to 80% |

TABLE B
POST-TRAUMATIC STRESS DISORDER

| | | MSM βᵢ | % |
|---|---|---|---|
| Function | Test, Method, Score or Scale | Weight | Improvement |
| Psychological | Trait Anxiety | 5 | Up to 80% |
| variables | State Anxiety | 5 | Up to 80% |
| | Self-esteem | 4 | Up to 70% |
| | Depression | 4 | Up to 70% |
| | Body image assessment | 4 | Up to 70% |
| | Attention | 4 | Up to 70% |
| | Memory | 4 | Up to 70% |
| | Personality traits | 4 | Up to 70% |
| Physiological | Cortisol | 5 | Up to 80% |
| variables | Catecholamines | 4.5 | Up to 75% |
| | Prolactin | 4.5 | Up to 75% |
| | Ghrelin | 4 | Up to 70% |
| | Leptin | 4 | Up to 70% |
| | Insulin | 4 | Up to 70% |
| | Sex hormones | 4 | Up to 70% |
| | Growth hormone (GH | 3.5 | Up to 65% |
| | Thyroxine | 3.5 | Up to 65% |
| Functional | Gait and Gait Speed | 3 | Up to 60% |
| variables | Balance | 4 | Up to 70% |
| | Mobility | 2.5 | Up to 50% |
| | Endurance | 3.5 | Up to 65% |
| | Flexibility | 2.5 | Up to 50% |
| | Reaction time (response | 4.5 | Up to 75% |
| | to external stimuli) | | |
| | Agility | 3 | Up to 55% |
| | Coordination | 4 | Up to 70% |
| Anthropometric | Muscle Strength and Tension | 2 | Up to 40% |
| assessments | Body Mass | 2 | Up to 40% |
| | Body Fat | 2 | Up to 40% |
| | Muscle Mass | 2 | Up to 40% |
| | Physical Performance | 2 | Up to 40% |
| | Waist/hip ratio | 2 | Up to 40% |
| | Waist circumference | 2 | Up to 40% |
| | Waist/height ratio | 2 | Up to 40% |
| | Bone mass | 2 | Up to 40% |
| | Residual mass | 2 | Up to 40% |
| | Weight Index | 3 | Up to 55% |
| Nutritional | Total energy profile | 2 | Up to 40% |
| variables | Number of meals and their | 2 | Up to 40% |
| | caloric distribution | | |
| | Macronutrients | 2 | Up to 40% |
| | Micronutrients | 2 | Up to 40% |
| | Nutrient density | 2 | Up to 40% |
| | Food variety | 2 | Up to 40% |
| | Fluid intake | 2 | Up to 40% |
| | Diet adherence | 2 | Up to 40% |
| | Biochemical nutritional status | 2 | Up to 40% |
| | Food consumption habits | 2 | Up to 40% |
| | Food consumption preferences | 3 | Up to 55% |
| Psychosocial | Hobbies | 2 | Up to 40% |
| variables | Daily physical activity | 4 | Up to 70% |
| | Type of lifestyle | 5 | Up to 80% |
| | (active to sedentary) | | |
| | Sleep/wake cycle patterns | 5 | Up to 80% |

TABLE C
COGNITIVE REJUVENATION

| | | MSM βᵢ | % |
|---|---|---|---|
| Function | Test, Method, Score or Scale | Weight | Improvement |
| Psychological | Trait Anxiety | 3 | Up to 60% |
| variables | State Anxiety | 3 | Up to 60% |
| | Self-esteem | 3 | Up to 60% |
| | Depression | 3 | Up to 60% |
| | Body image assessment | 3 | Up to 60% |
| | Attention | 5 | Up to 80% |
| | Memory | 5 | Up to 80% |
| | Personality traits | 4 | Up to 70% |
| Physiological | Cortisol | 2 | Up to 40% |
| variables | Catecholamines | 3 | Up to 60% |
| | Prolactin | 3 | Up to 60% |
| | Ghrelin | 3 | Up to 60% |
| | Leptin | 3 | Up to 60% |
| | Insulin | 3 | Up to 60% |
| | Sex hormones | 3 | Up to 60% |
| | Growth hormone (GH | 3 | Up to 60% |
| | Thyroxine | 3 | Up to 60% |
| Functional | Gait and Gait Speed | 3 | Up to 60% |
| variables | Balance | 4 | Up to 70% |
| | Mobility | 4 | Up to 70% |
| | Endurance | 4 | Up to 70% |
| | Flexibility | 4 | Up to 70% |
| | Reaction time (response | 4 | Up to 70% |
| | to external stimuli) | | |
| | Agility | 4 | Up to 70% |
| | Coordination | 4 | Up to 70% |
| Anthropometric | Muscle Strength and Tension | 2 | Up to 40% |
| assessments | Body Mass | 2 | Up to 40% |
| | Body Fat | 2 | Up to 40% |
| | Muscle Mass | 2 | Up to 40% |
| | Physical Performance | 2 | Up to 40% |
| | Waist/hip ratio | 2 | Up to 40% |
| | Waist circumference | 2 | Up to 40% |
| | Waist/height ratio | 2 | Up to 40% |
| | Bone mass | 2 | Up to 40% |
| | Residual mass | 2 | Up to 40% |
| | Weight Index | 3 | Up to 55% |
| Nutritional | Total energy profile | 2 | Up to 40% |
| variables | Number of meals and their | 2 | Up to 40% |
| | caloric distribution | | |
| | Macronutrients | 2 | Up to 40% |
| | Micronutrients | 2 | Up to 40% |
| | Nutrient density | 2 | Up to 40% |
| | Food variety | 2 | Up to 40% |
| | Fluid intake | 2 | Up to 40% |
| | Diet adherence | 2 | Up to 40% |
| | Biochemical nutritional status | 2 | Up to 40% |
| | Food consumption habits | 2 | Up to 40% |
| | Food consumption preferences | 3 | Up to 55% |
| Psychosocial | Hobbies | 2 | Up to 40% |
| variables | Daily physical activity | 4 | Up to 70% |
| | Type of lifestyle | 5 | Up to 80% |
| | (active to sedentary) | | |
| | Sleep/wake cycle patterns | 5 | Up to 80% |

TABLE D
OBESITY/OVERWEIGHT

| | | MSM βᵢ | % |
|---|---|---|---|
| Function | Test, Method, Score or Scale | Weight | improvement |
| Psychological | Trait Anxiety | 4 | Up to 70% |
| variables | State Anxiety | 4 | Up to 70% |
| | Self-esteem | 4 | Up to 70% |
| | Depression | 4 | Up to 70% |
| | Body image assessment | 4 | Up to 70% |
| | Attention | 2 | Up to 40% |
| | Memory | 2 | Up to 40% |
| | Personality traits | 3 | Up to 60% |
| Physiological | Cortisol | 4 | Up to 70% |
| variables | Catecholamines | 4.5 | Up to 75% |
| | Prolactin | 4.5 | Up to 75% |
| | Ghrelin | 3 | Up to 60% |
| | Leptin | 4 | Up to 70% |
| | Insulin | 4 | Up to 70% |
| | Sex hormones | 3 | Up to 60% |
| | Growth hormone (GH | 4 | Up to 70% |
| | Thyroxine | 3.5 | Up to 65% |
| Functional | Gait and Gait Speed | 5 | Up to 80% |
| variables | Balance | 4 | Up to 70% |
| | Mobility | 4 | Up to 70% |
| | Endurance | 4 | Up to 70% |
| | Flexibility | 4 | Up to 70% |
| | Reaction time (response | 4 | Up to 70% |
| | to external stimuli) | | |
| | Agility | 4.5 | Up to 75% |
| | Coordination | 4 | Up to 70% |
| Anthropometric | Muscle Strength and Tension | 4.5 | Up to 75% |
| assessments | Body Mass | 4.5 | Up to 75% |
| | Body Fat | 5 | Up to 80% |
| | Muscle Mass | 5 | Up to 80% |
| | Physical Performance | 4.5 | Up to 75% |
| | Waist/hip ratio | 4.5 | Up to 75% |
| | Waist circumference | 4.5 | Up to 75% |
| | Waist/height ratio | 4.5 | Up to 75% |
| | Bone mass | 4.5 | Up to 75% |
| | Residual mass | 4 | Up to 70% |
| | Weight Index | 4.5 | Up to 75% |
| Nutritional | Total energy profile | 4.5 | Up to 75% |
| variables | Number of meals and their | 4.5 | Up to 75% |
| | caloric distribution | | |
| | Macronutrients | 4.5 | Up to 75% |
| | Micronutrients | 4.5 | Up to 75% |
| | Nutrient density | 4.5 | Up to 75% |
| | Food variety | 4.5 | Up to 75% |
| | Fluid intake | 4.5 | Up to 75% |
| | Diet adherence | 5 | Up to 80% |
| | Biochemical nutritional status | 5 | Up to 40% |
| | Food consumption habits | 5 | Up to 80% |
| | Food consumption preferences | 5 | Up to 80% |
| Psychosocial | Hobbies | 3.5 | Up to 65% |
| variables | Daily physical activity | 2.5 | Up to 50% |
| | Type of lifestyle | 5 | Up to 80% |
| | (active to sedentary) | | |
| | Sleep/wake cycle patterns | 5 | Up to 80% |

The scale of values referring to both the caloric and nutritional profile is inverted with respect to the parameters evaluated so far; that is, instead of estimating “high level” as undesirable or significant values of worsening or absence of improvement, “suboptimal level” will be applied with the same meaning; that is, it does not reach the minimum nutritional parameters required to verify an estimable evolution.

The SMM Scale is calculated using a Napierian logarithm applied to the different parameters of the different psychological processes, motor functions, anthropometric assessments, and nutritional profiles. These parameters can be both existing external scales and measurements of tests performed and they represent the various sub-scores measured as a real scalar number. Each of the parameters has its own range of allowed values that varies from its value “min” to “max”. In one embodiment, a function is used that relates any sub score value [symbol] (xᵢ) (xi) of the “ ” scalexᵢ to a real number between −1 and +1, with 0 being the optimal value for that score i, −1 being the worst value below the optimal, and +1 being the worst value above the optimal to normalize the SMM Scale to a range from minimum to maximum in accuracy level. This function may vary depending on the complexity and objectives established for it (median, deviation, etc.). For example, when the median value is considered the optimal target, this function would be:

$$\text{?} ( {x}_{i} ) = 1 + \frac{2 \times ( {x}_{i} - {min}_{i} )}{{max}_{i} - {min}_{i}}$$

In one embodiment, the function could have a more complex form, such as:

$$\text{?} ( {x}_{i} ) = \{ \begin{matrix}{x}_{i} \leq 15 \to 0 \\ {x}_{i} > 15 \to 1 + \frac{2 \times ( {x}_{i} - {min}_{i} )}{{max}_{i} - {min}_{i}}\end{matrix}$$

To normalize the values, a weight βᵢ is assigned to each sub-score is attributed to emphasize the strength of the parameters of the different psychological processes, motor functions, anthropometric assessments, and nutritional profiles in the result obtained by the SMM Protocol. This weight, attributed according to the importance of each parameter or sub-score, must add a total equal to 1 to validate according to these weights that percentage corresponds to the level of weight in the final SMM Scale. Its mathematical function is detailed below:

$${\sum }_{i = 1}^{N} {β}_{i} = 1 .$$

In one embodiment, the SMM Protocol establishes the following list of Weights established for scales and different evaluation methods based on different behavioral functions to be modified. Different Weights can be assigned by an operator according to the behavioral function to be modified.

Next, the exponential of the sum of all the Napierian logarithms of the squared function of the sub-scores is calculated by adding one to normalize it to positive values and multiplying the function by each weight associated with the sub-score. Finally, the result of the exponential expression is subtracted from the value of 1 to obtain the result of the final SMM Scale that fluctuates between the values of [−1,1]. All timescales made during the SMM Protocol sessions aim to be close to the optimal value of a “0” scale according to the following formula:

$$\begin{matrix}Scale MSM = {e}^{( {\sum }_{i = 1}^{N} ln [ 1 + {( \text{?} ( {x}_{i} ) )}^{2} ] \times {β}_{i} )} - 1 \\ score ϵ ℝ ( 0 , 1 )\end{matrix}$$

where:
N: different sub-scores measured as a scalar real number.
minᵢ: minimum valid number for the subscaleᵢ
maxᵢ: maximum valid number for the subscale i
βᵢ: weight assigned to the subscale so i
[symbol] (xᵢ): a function that relates any value of sub score to a real number between −1 and +1, being 0 the optimal value for that score, −1 the worst value below the optimal and +1 the worst value over the optimal. xᵢi

### EXAMPLES

### Weight Control Example Applicability of the Optimization Artificial Intelligence Model.

A description of an example using the Adaptive Stimulation Program Orchestration System follows:

A neurostimulation program with the aim of reducing weight in people with obesity is configured in the Adaptive Stimulation Program Orchestration System so the result is defined as the percentage of body fat measured by impedance and the calculation of the Body Mass Index using the formula:

$$BMI = \frac{Weight in kg}{Height in {meters}^{2}}$$

Height is provided by the user at the beginning of the SMM protocol and considered to have a constant value. Weight and body fat percentage are measured with an electronic body composition device connected via Bluetooth to a portable device running an SP. The portable device sends a message to the SMM protocol user requesting that the result measurement be updated every 48 hours.

Certain activities are designed to incorporate a variety of multisensory stimuli, such as:

### Activity A: Aversive Stimulus of Junk Food.

This activity comprises the combination of a conditioned stimulus of the visualization of eating junk food with VR glasses accompanied by one of the following aversive stimuli: tactile pressure on the abdomen at a level that produces discomfort for the user provided with an interactive use, or a sharp and loud noise that produces discomfort for the user or an auditory stimulus consisting of a register that generates negative emotions. mood (former people crying) or a visual stimulus consisting of a photograph of the user where deformation or aging filters have been applied.

Each of these aversive stimuli is prepared with different intensities, for example there are different levels of tactile abdominal pressure levels, different volumes and frequencies of loud noise, different intensities of people crying recordings and different levels of aging filters applied to the user's photo.

A set of different durations of aversive stimuli are also configured, from milliseconds to 5 minutes.

### Activity B: Stimulus to Reinforce Healthy Foods.

This activity comprises the combination of a conditioned stimulus of the visualization of eating healthy foods with VR glasses accompanied by one of the following reinforcement stimuli: relaxing tactile vibrations in the back provided with an interactive use, or a pleasant auditory stimulus consisting of a register that generates a positive emotional mood (ex-positive messages) or a visual stimulus that consists of a photograph of the user in a good fit or younger by using filters.

Each of these reinforcement stimuli are prepared with different intensities and durations are configured from milliseconds to 5 minutes.

### Activity C: Exercise Reinforcement Stimulus.

This activity comprises the combination of a conditioned stimulus of the realization of a program of guided exercises with the help of VR glasses accompanied by one of the following reinforcement stimuli: relaxing tactile vibrations in the back provided with an interactive use, or a pleasant auditory stimulus consisting of a register that generates a positive emotional mood (ex-positive messages) or a visual stimulus that consists of a photograph of the user. in a good fit or younger by the use of filters.

Each of these reinforcement stimuli are prepared with different intensities and durations are configured from milliseconds to 5 minutes.

Embodiment 1: Anxiety description: adult person with the following symptoms: palpitations, tachycardia, choking sensation, with chest tightness.

Objective: to reduce or eliminate states and symptoms of anxiety in which you feel threatened by internal or external danger.

### SMM Protocol to Follow:

Video 1 (30 seconds). Introduction to the SMM Protocol to know the objective and generate more strength or motivation. Explanatory, descriptive, and motivational audio-video. The objective is to facilitate the individual's immersion in the project in a proactive way. Throughout the audio-video, the association of images, music and smells will be projected simultaneously to the “voice-over”, inviting relaxation and, therefore, to activate the capacity of concentration and focus.
Video 2 (2 minutes). Relaxation. The session begins with images of a forest in which the person is walking in first person through it; during this time relaxing music, soft and pleasant smells are synchronized (one minute). We would simulate walking through the forest as realistically as possible (the rustling of branches with our footsteps, wind, birds singing, smell of forest, sunlight or raindrops on the face, etc.). Breathing at different rhythms will be done during the time of the video. The images will be synchronized with pleasant smells, forest sounds, breathing sounds, tactile stimulation.
Video 3 (2 minutes). Physical exercise and breathing. In this occasion, the user in first person will be reflected performing high intensity physical exercises at different frequencies, integrated with breathing rhythms. During the exercises, pleasant smells and tactile stimulation will be synchronized with the rhythm of the movements and breathing. The exercise ends by drinking a glass of water—in first person-synchronized with taste and tactile stimulation.
Video 4 (2 minutes). Presentation of the images of their own anxiety attacks, taken from the clinical history that is done to the user. The user will observe in first person the images and sounds, in different planes, from far to near, with increased ocular motility during the exposure of these images and alternating positive images (right hemifield) with images associated with stress (left hemifield). As it gets closer, the multisensory intensity will be increased (olfactory, visual-colors- and auditory intensities, as well as body tactile stimulation). During this preparation, smell, taste, sounds of food preparation and tactile stimulation will be synchronized.
Video 5 (7 minutes). Context based on anxiety states. The individual—in first person—observes the context in which the events that provoke his high levels of anxiety have developed. During the exercise the multisensory stimuli associated with the images of the anxiety episode will be synchronized.
Video 6 (2 minutes) Relaxation. Finally, the session will end with video 2 (interaction with the forest) synchronized with breathing rhythms and multisensory stimulation.

Embodiment 2: Post-traumatic stress disorder description: war veteran who experiences flashbacks of the traumatic event repeatedly, accompanied by physical symptoms such as palpitations and difficulty falling asleep.

Objective: diminish or cancel the psychological and physiological effects of situations or events that cause stress to relive.

### SMM Protocol to Follow:

Video 1 (30 seconds). Introduction to the SMM Protocol to know the objective and to generate more strength or motivation. Explanatory, descriptive, and motivational audio-video. The objective is to facilitate the immersion of the individual in the project in a proactive way. Throughout the audio-video, images, music, and associated smells will be projected simultaneously to the “voice-over”, inviting relaxation and, therefore, to activate the capacity of concentration and focus.
Video 2 (1 minute). Relaxation. The session begins with images of a forest in which the person is walking-in first person-; during this time relaxing music, soft and pleasant smells are synchronized (one minute). We would simulate walking through the forest as realistically as possible (the rustling of branches with our footsteps, wind, birds singing, forest smell, sunlight, or raindrops on the face, etc.). Breathing at different rhythms will be done during the time of the video. The images will be synchronized with pleasant smells, forest sounds, breathing sounds, tactile stimulation.
Video 3 (2 minutes). Physical exercise and breathing. In this occasion, the user in first person will be reflected performing high intensity physical exercises, at different frequencies, integrated with breathing rhythms. During the exercises, pleasant smells and tactile stimulation will be synchronized with the rhythm of the movements and breathing. The exercise ends with drinking a glass of water—in first person—synchronized with tactile stimulation and taste.
Video 4 (2 minutes). Presentation of images typical of post-traumatic stress. The user will observe in first person the images and sounds, in different planes, from far to near, with increased ocular motility during the exposure of these images and alternating positive images (right hemifield) with images associated with stress (left hemifield). As it gets closer, the multisensory intensity will be increased (olfactory and visual intensity-colors- and auditory, as well as body tactile stimulation). Smell, taste, sound, and tactile stimulation will all be synchronized during this preparation.
Video 5 (8 minutes). Context based on post-traumatic stress. The individual—in the first person—observes the context in which the events that provoke post-traumatic stress, taken from the clinical history that is done to the user, have developed. During the exercise the multisensory stimuli associated with the images of the anxiety episode will be synchronized.
Video 6 (2 minutes). Relaxation. Finally, the session will end with video 2 (interaction with the forest) synchronized with breathing rhythms and multisensory stimulation.

Embodiment 3: Overweight description: overweight adult person with a body mass index (BMI) of 28 who is sedentary.

Objective: Healthy lifestyle habits and consequently weight loss focused on fat loss while avoiding the loss of muscle mass and even increasing it

### SMM Protocol to Follow:

Video 1 (30 seconds). Introduction to the SMM Protocol to know the objective and generate more strength or motivation. Explanatory, descriptive, and motivational audio-video. The objective is to facilitate the immersion of the individual in the project in a proactive way. Throughout the audio-video, there will be a simultaneous projection of “voice-over” associated with images, music, and smells that encourage relaxation and, as a result, activate the ability to concentrate and focus.
Video 2 (1 minute). Relaxation. The session begins with images of a forest: the individual is walking in first person through it, during this time relaxing music, soft and pleasant smells are synchronized. We would simulate walking through the forest as realistically as possible (the rustling of branches with our footsteps, wind, birds singing, smell of forest, sunlight, or raindrops on the face, etc.). Breathing at different rhythms will be done during the time of the video. The images will be synchronized with pleasant smells, forest sounds, breathing sounds, tactile stimulation.
Video 3 (1 minute). Physical exercise and breathing. On this occasion, the user will be reflected in the first person while performing high-intensity physical exercises at various frequencies that are integrated with breathing rhythms. During the exercises, pleasant smells and abdominal tactile stimulation will be synchronized with the rhythm of the movements and breathing. The exercise ends with drinking a glass of water—in first person—synchronized with taste and tactile stimulation.
Video 4 (2 minutes). Presentation of the healthy food to be “ingested” and preparation of the recipe of the healthy food to be “ingested”. The user will observe the food in first person, from a distance to up close. The multisensory intensity will increase as he/she gets closer (olfactory and visual intensity (colors), auditory, and tactile stimulation in the abdomen). The user in the first person will observe the preparation of the recipe. During this preparation, smell, taste, sounds of food preparation and tactile stimulation will be synchronized.
Video 5 (10 minutes). Healthy food intake. The individual—in first person-sits at the table, brings the plate and cutlery, and begins, bite by bite, to taste the menu at a slow pace (about 20 seconds chewing food in the mouth). During the exercise, sounds of swallowing, chewing and ingestion of liquids, an image of himself thin, palatable taste stimuli on the tongue, olfactory stimuli according to the smell of the food, tactile stimuli will be synchronized at the moment when he brings the food to his mouth. The ingestion process will end before the plate is empty.
Video 6 (1 minute). Relaxation. Finally, the session will end with video 2 (interaction with the forest), synchronized with respiratory rhythms and multisensory stimulation.

Embodiment 4: Cognitive Rejuvenation Description: Elderly Person with Memory, Language, Thinking and Decision Making.

Objective: establish the four basic pillars (mental state, cognitive, diet and exercise) to generate an adequate lifestyle to promote longevity.

### SMM Protocol to Follow:

Video 1 (30 seconds). Introduction to the protocol to know the objective and generate more strength or motivation. Explanatory, descriptive, and motivational audio-video. The objective is to facilitate the immersion of the individual in the project in a proactive way. Throughout the audio-video, images and associated music and smells will be projected simultaneously with the “voice-over”, inviting relaxation and, therefore, to activate the ability to concentrate and focus.
2. Video 2 (2 minutes). Relaxation. The session begins with images of a forest in which the person is walking in the first person; during this time relaxing music, soft and pleasant smells are synchronized (one minute). We would simulate walking through the forest as realistically as possible (the rustling of branches with our footsteps, wind, birds singing, smell of forest, sunlight or raindrops on the face, etc.). Breathing at different rhythms will be done during the time of the video. The images will be synchronized with pleasant smells, forest sounds, breathing sounds, tactile stimulation.
3. Video 3 (2 minutes). Physical exercise and breathing. In this occasion, the user in first person will be reflected performing high intensity physical exercises, at different frequencies, integrated with breathing rhythms. During the exercises, pleasant smells and tactile stimulation will be synchronized with the rhythm of the movements and breathing. The exercise ends with drinking a glass of water in first person synchronized with taste and tactile stimulation.
4. Video 4 (2 minutes). Presentation of images and sounds typical of scenes of everyday life. The user will view the images in first person, on various planes ranging from far to near. The multisensory intensity will increase as he/she gets closer (olfactory and visual intensity (colors), auditory, and body tactile stimulation). During this preparation, smell, taste, sounds of food preparation and tactile stimulation will be synchronized.
5. Video 5 (7 minutes). Context based on scenes of daily life. The individual—in first person—observes the context in which the events of daily life have developed. During the exercise, the multisensory stimuli associated with the images of the anxiety episode will be synchronized.
6. Video 2 (2 minutes). Relaxation. Finally, the session will end with video 2 (interaction with the forest) synchronized with breathing rhythms and multisensory stimulation.

While the invention has been described in reference to certain embodiments, it should be understood by those skilled in the art that various changes in form and detail may be made therein without departing from the scope and spirit of the invention encompassed by the claims. Numerous modifications, changes, variations, substitution and equivalents will be apparent to those skilled in the art without departing from the spirit and scope of the invention as described in the claims.

What is claimed is:

1. A computer-controlled method of neurocognitive learning for modification of behavioral functions in a human, comprising:
determining a behavioral function of a human subject to be modified;
measuring values of initial control variables of the human subject, wherein the initial control variables comprise one or more of medical control variables, psychological control variables, psychosocial control variables, functional control variables, sensory control variables and combinations thereof;
conducting one or more sequential training sessions with the human subject, wherein the human subject is presented with one or more sensory stimuli, the one or more sensory stimuli using brain frequency bands comprising visual stimuli, auditory stimuli, tactile stimuli, taste stimuli, olfactory stimuli and combinations thereof.
measuring one or more of medical control variables, psychological control variables, psychosocial control variables, functional control variables, sensory control variables and combinations thereof after each sequential training session;
comparing values of the one or more of medical control variables, psychological control variables, psychosocial control variables, functional control variables, sensory control variables and combinations thereof measured after each sequential training session to the values of the initial control variables;
comparing values of the one or more of medical control variables, psychological control variables, psychosocial control variables, functional control variables, sensory control variables and combinations thereof to the values of the one or more of medical control variables, psychological control variables, psychosocial control variables, functional control variables, sensory control variables and combinations thereof measured in a prior sequential training session;
analyzing the comparison of the one or more of medical control variables, psychological control variables, psychosocial control variables, functional control variables, sensory control variables and combinations thereof to previous values;
analyzing using artificial intelligence software changes to sensory stimuli input for subsequent training sessions; and
conducting subsequent training sessions until the artificial intelligence software determines that no further changes to sensory stimuli input are recommended.

2. The computer-controlled method of claim 1, wherein the sensory stimuli comprise brain frequency bands, wherein the sensory stimuli further comprise one or more of visual stimuli: auditory stimuli; touch stimuli; taste stimuli; smell stimuli; image stimuli; and combinations thereof.

3. The computer-controlled method of claim 2, wherein the one or more image stimuli are presented during an initial training session or a subsequent training session using one or more of virtual reality, augmented reality and extended reality.

4. The computer-controlled method of claim 3, further comprising a readable computer memory and database, wherein one or more computer readable files comprising a multisensory stimulation catalog are stored in the readable computer memory and database, wherein further the one or more computer readable files store information regarding the sensory stimuli input to be presented to the human subject during the initial training session or any subsequent training session.

5. The computer-controlled method of claim 4, wherein the readable computer memory and database further comprises an artificial intelligence module, wherein the artificial intelligence module comprises a set of software modules programmed to generate random stimulation programs that identify combinations of sensory stimuli to be presented to the human subject during the initial training session and subsequent training sessions.

6. The computer-controlled method of claim 5, wherein the artificial intelligence module determines the order of execution, the temporality, and the intensity of the sensory stimuli to be presented to the human subject during the initial training session and subsequent training sessions.

7. The computer-controlled method of claim 6, wherein the sensory stimuli using brain frequency bands comprising delta, theta, alpha, beta and gamma frequencies are presented to the human subject during the initial training session and subsequent training sessions by use of one or more wearable devices, wherein the wearable devices are under control of the artificial intelligence module.

8. The computer-controlled method of claim 7, wherein the one or more wearable devices comprise one or more of reality glasses, headphones, smart clothes and combinations thereof.

9. The computer-controlled method of claim 6, wherein the random stimulation programs comprise one or more activities, wherein each of the one or more activities comprises one or more sensory stimuli using brain frequency bands.

10. The computer-controlled method of claim 9, wherein the one or more activities are executed by the artificial intelligence module in a specific pre-determined order and time sequence, wherein further the specific pre-determined order and time sequence is manually defined by a human operator.

11. A computer-controlled system for neurocognitive learning for modification of behavioral functions in a human, comprising:
a programmable computer, the programmable computer comprising one or more processors, one or more readable memories and one or more databases;
an artificial intelligence module stored in the one or more databases and in communication with the one or more processors;
one or more readable files comprising information regarding one or more sensory stimuli stored in the one or more databases; and
one or more wearable devices configured to deliver one or more sensory stimuli under control of the artificial intelligence module;
wherein the computer-controlled system for neurocognitive learning for modification of behavioral functions in a human performs the method of claim 1.

12. A computer-controlled method of developing a library of simulation programs with artificial intelligence methods for use in the system of claim 1, comprising:
defining a plurality of simulation programs, wherein each simulation program comprises a series of activities that are executed in a specific order, wherein each activity of the series of activities comprises one or more stimuli, wherein each of the one or more stimuli comprises a time duration, wherein further each of the one or more stimuli comprises an intensity level,
conducting the one or more activities on a human subject and thereafter measuring one or more results of conducting the one or more activities, wherein the results comprise a physiological effect of the one or more activities on the human subject;
thereafter determining if conducting the one or more activities on the human subject effects a behavioral function of the human subject;
thereafter adjusting the specific order, time duration, stimuli intensity level or combinations thereof of the one or more activities;
thereafter conducting the one or more activities as adjusted on the human subject and thereafter measuring one or more results of conducting the one or more activities as adjusted, wherein the results comprise a physiological effect of the one or more activities on the human subject;
thereafter determining if conducting the one or more activities as adjusted on the human subject effects a behavioral function of the human subject; and
storing the effectiveness of each of the one or more activities as adjusted on a behavioral function with demographic information of each human subject in a database,
wherein the results of conducting the one or more activities on a human subject are measured by hardware devices that are disposed on the human subject, wherein the hardware devices comprise virtual reality glasses, headphones, smart clothes, smart devices and combinations thereof,
wherein the one or more stimuli comprise auditory stimuli, visual stimuli, tactile stimuli, olfactory stimuli and combinations thereof,

13. The computer-controlled method of claim 12, wherein the one or more activities are adversive or reinforcing of the behavioral function of each human subject.

14. The computer-controlled method of claim 12, wherein particular activities to be presented to a human subject during the method of claim 1 are selected based on effectiveness of the particular activities for human subjects having similar demographic information stored in the database.
